(19) United States
(12) Patent Application Publication
(10) Pub. No.: US 2021/0282934 A1
(43) Pub. Date: Sep. 16, 2021

(54) INSERT FOR USE IN A WEAR COUPLE INCLUDING A SPHERICAL WEAR PARTNER

(71) Applicant: CeramTec GmbH, Plochingen (DE)

(72) Inventor: Mateusz Maria JUSZCZYK, Velden (DE)

(21) Appl. No.: 17/053,281

(22) PCT Filed: May 6, 2019
(86) PCT No.: PCT/EP2019/061525

§ 371 Date: Nov. 5, 2020

(30) Foreign Application Priority Data
May 7, 2018 (EP) 18171109.4

(51) Int. Cl. A61F 2/30

(52) U.S. Cl. CPC A61F 2/30734; A61F 2002/302; A61L 27/18; A61F 2002/30655; A61F 2310/00179; A61F 2002/30242

(57) ABSTRACT

The invention describes an implant for wear couples in endoprosthetics, comprising at least one shell, into which an insert, preferably a ceramic insert, is introduced. The insert has an outer side, with an outer face, and an inner side, and a hemispherical wear region for accommodating a spherical wear partner being formed on the inner side. The aim of the invention is to reduce the height of the implant as much as possible and to ensure that, e.g., the pelvic bone does not have to be milled down as much. According to the invention, the insert is therefore designed in the form of a ring or annular structure. In order to reduce friction between the spherical wear partner and the implant to a minimum, the implant has a specially designed inner geometry.

The invention relates to an implant, comprising a shell having an insert, for tribological pairing in endoprosthesis, the insert comprising an outside and an inside or inner surface, and a specially designed sliding region for receiving a spherical sliding partner being formed on the inner surface.

Hitherto, in endoprosthesis implants consisting of a metal shell and a ceramic half-shell insert were used. The metal shell is also designed as a half-shell and receives the ceramic insert. The sliding partner, the prosthesis head, is spherical and is received by the ceramic insert. Ceramic inserts for tribological pairing in hip replacement are hemispherical and cover approximately 50% of the prosthesis head. The central point of the sliding surface rests on the plane of the end face of the insert, or slightly thereabove or therebelow. The outside of the insert is divided into a plurality of regions. The region of the outside, on the equator, comprises a clamping surface which may be conical or cylindrical. By means of said clamping surface an operative connection to a shell, usually a metal shell, is established. The insert is inserted into the shell. This is carried out either in a manner already premounted following production, or only during implantation.

A further region of the outside, the rear face of the insert, which extends from the equator to the pole, is not in contact with the metal shell but, for reasons of stability, must have a minimum wall thickness.

In the case of this pairing, the load transfer between the femoral head and insert or acetabulum in the sliding surface takes place in a punctual or circumferential manner, since there is a positive clearance between the sphere diameter of the prosthesis head and the indentation diameter of the insert. In this case, the load is transferred to the insert from the femoral head in an axiparallel manner.

DE 10 2016 222 616 A1 discloses a ceramic annular insert which is introduced into the metal shell and comprises, on the inside, a hemispherical sliding surface for receiving the spherical sliding partner. The overall depth for the metal shell plus the annular insert is reduced, such that a less deep recess is required in the pelvic bone. Furthermore, there are no punctual loads, but rather strip-like loads having reduced maximum values, similar to the physiological load bearing capacity.

Proceeding therefrom, the object was that of providing an improved system for surgical use, the friction between the spherical sliding partner and the ceramic insert being reduced. Furthermore, the object consisted in developing an implant for endoprosthesis that is as cost-effective and stable as possible.

This object is achieved according to the invention by an insert according to the features of claim 1 and by an implant according to claim 12. Advantageous embodiments are specified in the dependent claims. Embodiments can be combined with one another as desired.

The implant is used, according to the invention, for receiving a sphere of a prosthesis head, i.e. a spherical sliding partner. The implant is retained in a stationary manner in the pelvic bone. The sphere of the prosthetic head and the insert form a tribological pairing. The sphere of the prosthesis head should be able to rotate in the insert. In this case, it should be possible to prevent the sphere of the prosthesis from springing out.

The implant according to the invention is formed of a shell or socket, into which the insert is introduced.

The shell can be a metal shell, preferably consisting of titanium and/or cobalt-containing and/or chromium-containing alloys, or a plastics shell, preferably consisting of polyethylene. The shell is used for fastening the implant in the bone. The shell is preferably made of a biocompatible metal. The insert is preferably ceramic, at least in part, preferably manufactured from a solid ceramic.

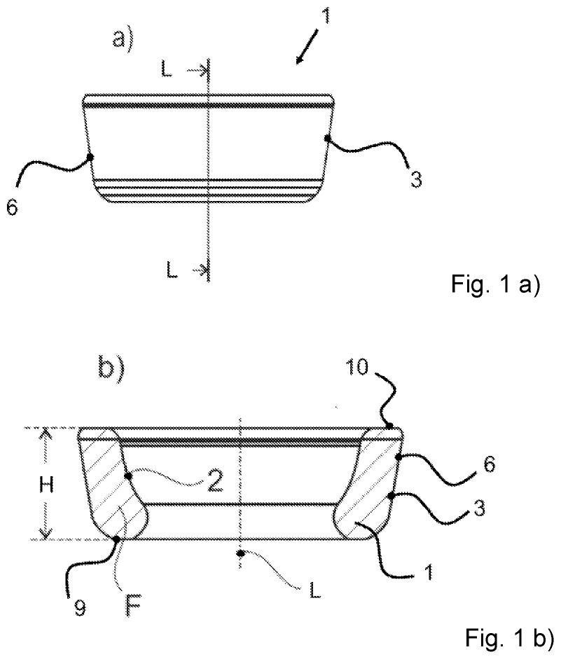

In the present case, an insert, preferably an annular insert (ring), is to be understood as a member that is formed of a cross-sectional surface F (see FIG. 1b) which rotates about an axis of rotation L (see FIG. 1b). The member comprises a concave inner surface and an outside. The shape of the outside can be formed in a manner deviating from the shape of the inside.

The insert comprises a first region, comprising an end face and an infeed zone which ensures the introduction of a sphere of a prosthesis head of the spherical sliding partner into the insert, and a second region which limits the reception of the sphere. In one embodiment, the insert comprises a half shell, the second region of which is closed. In a further embodiment, the insert corresponds to a ring, the second region of which, comprising a base surface and a discharge zone, is open. The circular opening of the first region, of the receiving region, has a diameter that is greater than the diameter of the opening of the second region. In this case, the circular opening of the second region of the annular insert is smaller than the diameter of the spherical sliding partner to be inserted, in order to prevent the spherical sliding partner, referred to in the following as KG, from slipping out.

The connection between the inner surface and the outside forms a transition and is preferably established by means of radii. The rounded transitions prevent sharp edges and corners, as a result of which the stability of the insert is improved. In addition, this facilitates the handling. These radii are preferably of a size of 0.5-2 mm. In the first region of the insert, the first transition of the inner surface to the outside comprises an end face.

In the case of the half-shell embodiment, the outside is closed. The surface development of the outside can correspond to a closed circle. The second region of the insert which is arranged opposite the first region is closed and comprises a closed base surface. The outside of said closed base surface is part of the outside of the insert. The maximum spacing between the first region, in which the end face is arranged, and the base surface, corresponds to the height H of the insert half shell. The inner surface of the closed base surface is part of the inner surface of the insert. The inner surface of the insert comprises a sliding region which is adjoined by the inner surface of the closed base surface. A half-shell embodiment of an insert comprises a first opening, and an infeed zone for introducing a sphere. The geometry of the inner surface of the closed base surface can correspond to a dome, a half-sphere, or a shape similar to a half-sphere.

The annular embodiment of the insert comprises a second opening opposite the first region. The diameter of said second opening is smaller than the diameter of the first opening of the first region. As a result, the introduction of the KG into the insert is made possible and is limited. The surface development of the outside of the annular insert corresponds to a ring. As a result of the opening, which is arranged opposite the first region of the insert, this comprises a second transition between the inner surface and the outside. This is located in the second region and limits the height of the insert. The transition between the inner surface and the outside comprises a base surface. The maximum spacing between the end face and the second transition or the base surface corresponds to the height H of the annular insert.

The rounded first transition of the inner surface of the insert, as far as the start of the sliding region, is referred to as the infeed zone, the rounded second transition of the inner surface of the insert, as far as the start of the sliding region, is referred to as the discharge zone.

The inner surface is designed so as to be rotationally symmetrical, at least in part. The outside and/or outer surface of the insert, preferably of the annular insert, can deviate from the rotational symmetry in regions. The height H (see FIG. 1b) of the insert is understood to be the extension thereof along the axis of rotation L. In the annular embodiment, the height H is substantially smaller compared with the half-shell embodiment. The outside of the insert corresponds to the side which faces the bone in which the insert is intended to be inserted. The outer surface is a region of the outside and is used for fastening the insert in the shell, preferably a metal shell. The size of the outer surface can correspond to the size of the outside. It can also be designed so as to be smaller. The outer surface can assume various shapes, or be divided into a plurality of regions or individual surfaces which are interconnected or delimited from one another. The design of the individual surfaces can be the same or different.

The insert according to the invention is designed as a half shell or ring such that it interacts with spheres and shells according to the prior art, the functionality being ensured. The insert has a wall thickness of at least 3 mm, in order to ensure the stability. The maximum wall thickness of the insert depends on the sinter properties of the material used, and is in the region of 15 mm, preferably a maximum of 15 mm. The height H of the annular insert is preferably 5-20 mm. Depending on the situation during use, an insert having the appropriate geometric dimensions is used.

The inner surface of the insert comprises a sliding region on which the KG is intended to rotate. The sliding region of the insert is concave and corresponds to a portion of a surface of a rotation member.

The rotation member is a spindle torus which is described by a circle 108 that rotates about an axis of rotation. The spacing A of the axis of rotation from the center point M′/M″ of the circle is smaller than the radius r of the circle describing the torus. The center point straight lines L′ and L″ are located in parallel with the axis of rotation L. The torus describes, in the inside, a spindle 105 having a center point M which is located in the center of the straight line that describes the maximum longitudinal extension of the spindle 105 and rests on the axis of rotation. The points of intersection of the outer surface of the spindle 105 with the axis L are designated E and E′. In this case, the surface is the outer surface 106 of the spindle 105 thus described.

The portion 107 that describes the sliding region of the insert corresponds to the region between the two normal planes S and S′ which intersect the longitudinal axis L, which corresponds to the axis of rotation of the spindle torus, at points S1 and S2. Both points of intersection are located between E′ and M, i.e. in one half of the spindle 105, in the longitudinal extension. S1 can correspond to the center point M of the spindle 105. S2 is located between S1 and E′ or corresponds to E′.

Therefore, in the simplest embodiment thereof the insert has an inside geometry which corresponds to a portion of the spindle of a spindle torus, the region of the inner surface that is located between the end face and the base surface being concave, and it being possible for the geometry of the outside to deviate from the rotational symmetry. The sliding region of the inner surface is therefore not hemispherical, i.e. does not correspond to a portion of a sphere. The sliding region corresponds to the portion 107 of the outer surface 106 of a spindle 105. The portion 107 is located in one half of the spindle 105, along the longitudinal axis thereof, and does not exceed the center point M of the spindle on the longitudinal axis L of the spindle 105. In the direction of the end face, the sliding region of the insert has a maximum diameter D1 at the first opening thereof. At the second opening, the sliding region has a minimum diameter D2. The diameter D1 of the insert is larger than the diameter D2. The diameters of the spindle 105 between D1 and D2 reduce in size in the direction D2.

The inside geometry according to the invention ensures the movability of the KG, i.e. a sphere or a sphere portion of the prosthesis head. The diameter D1 of the first opening of the insert is larger than the outside diameter of the KG which is introduced into the insert. The diameter D2 is smaller than the outside diameter of the KG. The smallest diameter of the infeed zone is preferably larger than D1.

In one embodiment, S2 corresponds to the point E′. If S2 corresponds to E′, the insert is a half shell. In this embodiment of the half-shell insert, the diameter D2=0, i.e. no second opening is present.

In a further embodiment, the insert is a half shell and S2 is located above E′ on the axis L. In this embodiment, the inner surface is flattened in the region E′. The height H of the insert reduces as a result. In this embodiment of the half-shell insert, the geometry of the inner surface of the closed base surface deviates from the geometry of the spindle. In this case, care is preferably taken that the flattened inner surface of the closed base surface should be designed such that it does not influence the geometry of the contact line, and sufficient space is provided for the KG, in order not to create any point-based friction. This is then a hemispherical or preferably further flattened inner surface of the closed base surface.

In a preferred embodiment, S2 is located above E′ on the axis L, and the discharge zone adjoins the sliding region at D2. The insert is then a ring. In the annular embodiment, D2 is smaller than the radius of the KG to be inserted, in order to prevent falling out.

The KG thus rotates in the non-hemispherical sliding region of the inner surface, the sliding region corresponding to a portion 107 of half a spindle of a spindle torus in the longitudinal extension.

The height H_{G} of the sliding region corresponds to at least 20% and at most 80% of the diameter of the KG to be inserted, and preferably 50-95% of the height H of the insert.

The height of the sliding region corresponds to the extension in the longitudinal direction, i.e. along the axis of rotation L. The height H_{G} preferably corresponds to at least 25%, particularly preferably at least 30%, and preferably at most 70%, particularly preferably at most 60%, of the diameter of the KG to be inserted. For an annular insert, the height H_{G} is in particular at most 50% of the diameter of the KG.

In one embodiment, the KG has a diameter of 5-70 mm, preferably 6-64 mm. KG for human artificial joints have a diameter of 20-70 mm, preferably 22-64 mm, and for animal artificial joints KG having a diameter of 5-20 mm, preferably 6-19 mm, are used. As a result, in this embodiment an insert in which a KG having a diameter of 5 mm is intended to be inserted has a sliding region having a height of at least 1 mm and at most 4 mm.

Furthermore, in one embodiment the height H_{G} of the sliding region (2) corresponds to at least 20%, preferably at least 35%, particularly preferably at least 50%, and at most 95%, of the height H of the half-shell or annular insert. The discharge and infeed zone of an annular insert are not part of the sliding region. The infeed zone of the half-shell insert, and the inner surface of the closed base surface having a possible flattening, are not part of the sliding region. In the mounted state, the KG preferably does not touch the infeed and inner surface of the closed base surface of the half-shell insert.

With respect to the geometry of the spindle, the following conditions preferably apply:
A is the distance between L and L″ or the horizontal distance from the center point to the axis of rotation.
r is the radius of the circle describing the spindle torus.
r_{P} is the radius of the spherical sliding partner, i.e. the radius of the prosthesis head.
C is the clearance and complies with formula I:

C=(r−rₚ)*2 (formula I).

In one embodiment, the clearance corresponds to the sum of the maximum deviations, specified in production, of the extensions of the prosthesis head (of the radius r_{P}) and of an insert that has a hemispherical sliding region and is suitable for the prosthesis head. In a particular embodiment, C>10 μm, preferably >25 μm, particularly preferably ≥50 μm, and <500 μm, preferably <350 μm, and particularly preferably ≤280 μm.
The radius r of the circle 108 describing the spindle torus is larger than the radius r_{P} of the KG.

KG is in contact with the sliding region and slides thereon; KG is preferably in linear contact with the sliding region of the insert.

The contact line corresponds to a circular line in the sliding region, i.e. in the portion 107 on the outer surface 106 of the spindle 105 of the spindle torus. This line corresponds to the line of intersection of a sectional plane 111 through the spindle 105 in the region between S and S′. In the case of a firmly specified radius r_{P} of the prosthesis head and a firmly specified clearance, the diameter of the annular contact or the annular contact line can be influenced by changing the distance A. As a result, the angle α which between the longitudinal axis L of the spindle and the straight line, which connects the center point of the spherical sliding partner to a point 110 on the contact line, can be influenced. If α increases, the contact line is oriented in the direction of the infeed zone of the insert. If α decreases, the contact line is oriented in the direction of the base surface or discharge zone. Point contact would exist in a hemispherical insert if α=0. Since the insert according to the invention in the half-shell shape has a spindle shape, the sphere cannot touch the point of intersection E′.

In one embodiment of the annular insert the contact line is located in the lower half of the height H of the insert, i.e. in the half of the insert facing the second region. Viewed from the base surface or the discharge zone, the contact line is thus in the range of 0-50% of the height. As a result, the dislocation, i.e. the prosthesis head springing out of the insert, is counteracted. The contact line is preferably between 10-40% and particularly preferably between 20-30% of the width of the insert, viewed from the base surface. Said contact line, arranged at a distance from the base surface, also allows for the formation of lubrication film, e.g. consisting of synovial fluid, which assists the sliding of the sphere in the insert.

In the annular embodiment thereof the insert according to the invention has a substantially smaller height, and thus a substantially smaller installation depth, compared with a conventional half-shell insert. The recess in the bone for the inserts can therefore be smaller. This allows for the use of an artificial insert in very small or thin bones, in particular hip bones, as frequently occur in teenagers or children or animals. An insert according to the invention having a reduced height makes it possible for the depth required for inserting the insert to be reduced to a minimum.

Preferably, the concave sliding region extends over 80%, particularly preferably over 95%, most particularly preferably over the entire inner surface of the insert, as a result of which a large portion or the entire inner surface is available for the tribological pairing.

The center point of the sliding region is preferably arranged on the plane of the end face, or slightly thereabove or therebelow, in the range of 0 to 2 mm.

In a further embodiment of the insert according to the invention, the insert, preferably also the sliding region, is formed so as to be extended on the portion of the insert, along the longitudinal axis. This means that the height H of the insert, and preferably also the height H_{G} of the sliding region, change over the periphery of the circle. In this case, the insert, preferably also the sliding region, is formed so as to be elevated/extended either in the direction of the prosthesis head, beyond the end face of the insert, and/or, in the case of an annular insert, beyond the base surface of the insert.

This enlargement of the insert, preferably of the sliding region, is referred to as cranial enlargement, and comprises just a part, a portion of the peripheral surface of the insert. As a result, the tendency for dislocation is reduced. In this case, the center of rotation is preferably located on or below the end face.

A region or portion of the insert that is arranged in the region of the infeed zone is referred to as the cranial elevation. The height H of the insert expands as a result. In one embodiment, this elevation also lengthens the sliding region.

A region or a portion of the insert that is arranged in the region of the discharge zone is referred to as a cranial lengthening. In one embodiment, this lengthening elevation also enlarges the sliding region.

In one embodiment the cranial enlargement of the insert is formed by a balcony-like protrusion or a shaped projection, the inside of which is a continuation of the receiving space described by the circle lines or of the inner surface of the insert. In this case, the protrusion preferably makes up′ of the surface described by said circle lines, on which surface the cranial elevation and/or lengthening is located.

In another embodiment of an insert according to the invention, the end face is not arranged in a plane. By means of the continuous slope of the end face and/or of the base surface (in the case of an annular insert) of the insert, the cranial enlargement is achieved. Proceeding from a position on the end face (or base surface), said end face (or base surface) rises continuously until it has reached its highest point after 180 degrees. Proceeding from this highest point, the end face (or base surface) then drops again continuously as far as the starting point thereof. As a result, the end face or base surface is arranged at a shallow angle with respect to the axis of rotation R. The shallow angle of the tilted end face thus arranged is 95 to 105 degrees, preferably 97 to 101 degrees, particularly preferably 99.5 degrees. In this case, the center point of the sliding region is on or below the end face. The continuous ascent in the end or base surface can also be in a range that is less than 180 degrees. The same applies for the descent. The ascent and decent are preferably of the same length, but can also be of different lengths.

Owing to the cranial elevation, the maximum height H′ of the insert deviates, in the region of the cranial enlargement, from the height H of the insert without the cranial enlargement. The following applies for the height of the insert: H′=H+x+y. In one embodiment, the cranial enlargement also leads to an elevation of the sliding region; this applies analogously for the height of the sliding region: H_{G}′=H_{G}+x_{G}=y_{G}.

The maximum extension of the cranial lengthening is denoted by x. This is the distance between the sectional plane S′ and the point Y′. The distance x thus describes the height difference of the points X′ and Y′ along the axis of rotation L.

The maximum extension of the sliding region of the cranial lengthening is denoted x_{G}.

The maximum extension of the cranial elevation is denoted y. This is the distance between the sectional plane S and the point Y. The distance y thus describes the height difference of the points X and Y along the axis of rotation L.

The maximum extension of the sliding region of the cranial elevation is denoted y_{G} and describes the height difference of points X_{G} and Y_{G}.

If x=y=0, no cranial enlargement exists.

If x>0 and y=0, a cranial lengthening exists. In this case, in addition, in a preferred embodiment x_{G}>0.

If x=0 and y=0 a cranial elevation exists. In this case, in a preferred embodiment the following additionally applies: y_{G}>0.

In a further embodiment, the following applies: x>0 and y>0, where x=/≠y and x_{G}=/≠y_{G}≥0.

The distances x and y are directly proportional, depending on the diameter of the sphere, to be used, of the prosthesis head, and the sum of x+y is preferably 2-20 mm, particularly preferably 3-15 mm.

In one embodiment, the cranial elevation follows the geometry of the spindle. That is to say that the portion of the torus that forms the lengthened region of the insert, optionally the lengthened sliding region, of the cranial elevation, is a continuation of the geometry of the spindle.

In another embodiment, an insert having a cranial enlargement no longer has any rotational symmetry along the axis of rotation L, in the region of the cranial enlargement. In one embodiment, the radii of the insert, optionally of the sliding region, of the cranial enlargement are not oriented to the geometry of a spindle.

The value of the radius that defines the sliding region can then deviate from the value of the radius that defines the elevation. The value of said radius (of the elevation) can preferably be smaller than or equal to

$$\frac{D 1}{2} .$$

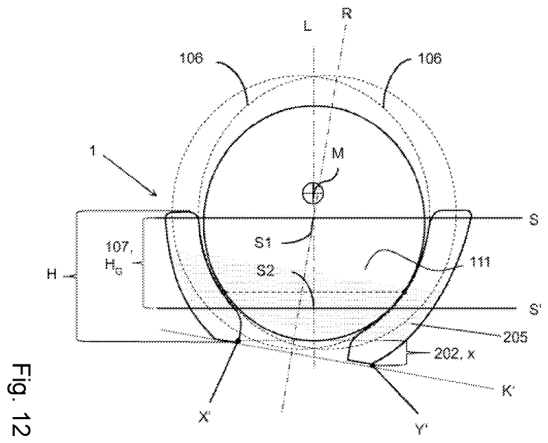

In this case, the cranial elevation must always fulfil the condition that the spherical sliding partner can still be inserted, i.e. the opening has a diameter that is larger than the diameter of the KG. The sectional plane through the spindle, which is located between a point X, which lies on the plane S and the outer surface of the spindle, and a further point Y, which lies opposite X and represents the maximum of the cranial elevation, must be of a diameter that corresponds at least to that of the region of the spherical sliding partner that is to be inserted. In this case, X lies on the opposing side from Y, i.e. a straight line K from X to Y intersects L. The preferred maximum cranial elevation of the insert results from a straight line K between X and Y, if this also intersects the center point of the spindle. This preferably applies analogously for a cranial elevation of the sliding region (shown in FIG. 11).

Particularly preferably, Y lies on the outer surface of the spindle. In this case, the inner surface further corresponds to a portion of a spindle, the part of the implant that completely surrounds the sliding partner in a circular manner corresponds to the portion of half of the spindle along the axis of rotation L thereof, and this part does not exceed the center point of the longitudinal axis of the spindle.

In one embodiment of the cranial lengthening, the radii of the implant, optionally of the sliding region, of the cranial enlargement are not oriented to the geometry of a spindle. The value of the radius that defines the sliding region can then deviate from the value of the radius that defines the lengthening. The value of said radius (the elevation) can be smaller than or equal to

$$\frac{D 2}{2} .$$

In a further embodiment, the portion of the torus that forms the lengthened sliding region is a continuation of the geometry of the spindle.

In this case, the cranial lengthening must always fulfil the condition that the spherical sliding partner still cannot fall out, i.e. the opening has a diameter that is smaller than the diameter of the KG. The sectional plane through the spindle, which is located between a point X′, which lies on the plane S′ and the outer surface of the spindle, and a further point Y′, which lies opposite X′ and represents the maximum of the cranial lengthening, must be of a diameter that is smaller than the diameter of the spherical sliding partner. In this case, X′ lies on the opposite side from Y′, i.e. a straight line K′ from X′ to Y′ intersects L (shown in FIG. 12). In this case, Y′ is particularly preferably also located on the outer surface of the spindle. This preferably applies analogously for a cranial lengthening of the sliding region.

In an ideal embodiment, the outside is conical, at least in a portion, preferably in a circumferential portion (around the axis of rotation). The design of the outside can deviate, in part or completely, from this ideal embodiment, since the outside of the insert corresponds to the side which is connected to the shell, preferably the metal shell.

At least one clamping surface is arranged on the outside of the insert. Said clamping surface is used for the purpose of anchoring in the shell. The insert is connected to the shell in a form-fitting, preferably force-locking, particularly preferably frictional manner. The outside of the insert is preferably rotationally symmetrical. The outside comprises an axis of rotation R and is formed conically, at an angle to the axis of rotation R. In other words, there is an acute angle, preferably between 10° and 20°, particularly preferably between 18° and 18.5°, between the axis of rotation and the outside. A conical insert is thus formed, the outside dimension of which in the second region is smaller than that in the first region. The clamping surface is arranged on the outside and can encompass the entire outside. Embodiments are also possible in which the shape of the clamping surface deviates from the shape of the outside, and comprises sub-regions or portions of the outside. The insert is form-fittingly connected to the shell by means of said clamping surface.

In a further embodiment, the outside of the insert can be symmetrical, and the acute angle is then 0°. The force-fitting connection, preferably the frictional connection, between the insert and the shell is then achieved by means of an interference fit.

In a preferred conical or cylindrical embodiment, the axis of rotation R is in parallel with the axis of rotation L of the spindle, and it particularly preferably corresponds to the axis of rotation L.

In a further embodiment, the axis of rotation L does not correspond to the axis of rotation R of the conical or cylindrical outside of the insert, preferably in the case of an annular insert having a cranial lengthening. Said axis of rotation R preferably intersects the axis of rotation L, particularly preferably in the region of the sliding region. The axis of rotation R is preferably arranged such that it is perpendicular to and intersects the straight line K′ which interconnects the maximum extension of the base surface of the annular insert with and without a cranial lengthening, in points X′ and Y′. If an insert of this kind is inserted into a conventional shell, the cranial lengthening appears as a cranial elevation, and the inside geometry corresponds to a spindle tilted away from the cranial elevation (shown in FIG. 12).

A joint having an annular implant according to the invention comprises a free space between the inside of the shell and the surface of the sphere of the prosthesis head. This ensures the freedom of movement of the joint.

In one embodiment of an annular insert according to the invention, the circumferential clamping surface is interrupted by recesses. The recesses are arranged along the width of the insert and connect the end face to the base surface. They may be arranged so as to be in parallel with the axis of rotation. These openings, which are formed by said recesses, allow for fluid to flow out, from the gap between the inside of the shell and the surface of the sphere of the prosthesis head. The recesses can be in the form of notches or tangential cuts, which extend over the entire width of the insert. The at least two recesses are preferably arranged symmetrically on the clamping surfaces of the insert.

The wall thickness of the insert is at least 3 mm. This minimum wall thickness of 3 mm is also provided in the region of the maximum extension of the recesses, i.e. also at the thinnest points of the insert. As a result, the stability of the insert can be ensured.

In a preferred embodiment of an annular insert, the insert preferably has a height H of 5-20 mm, particularly preferably 10-15 mm. At this height H, little space is required and, surprisingly, the clamping force generated by the form-fitting connection, preferably the force-fitting connection, particularly preferably the frictional connection, is nonetheless sufficient for establishing a secure connection between the annular insert and the shell. As a result of the reduced height H, the annular insert according to the invention is of approximately only half the height H of a conventional insert which is designed as a half shell. As a result thereof, it is possible to use a shell, the semicircular arc of which is less high, or the portion thereof, of the semicircular arc, which is located below the second region of the annular insert, is flatter. The only requirement is the free movement of the sphere of the prosthesis head. The sphere of the prosthesis head slides in the annular insert and does not touch the shell. Owing to the low width of the annular insert, in one embodiment the shell for receiving the annular insert is flatter than in the case of use of a conventional insert. As a result, less space is required for inserting an implant according to the invention. This protects the patients' bones.

In one embodiment of the implant according to the invention, recesses are provided, for example in the form of bores or holes in the shell. As a result, the shell can be fastened in or on the bone by means of fastening means, for example screws.

In the case of the insert having the half-shell design, these bores are preferably arranged in the shell such that, when the annular insert is mounted, they are accessible for the purpose of fastening in the bone. Since the annular insert is annular, at least a portion of the inside of the shell is also accessible, even when the insert is already mounted. As a result, when the bores are arranged accordingly, the shell can also be fastened to a mounted annular insert, on the bone, using fastening means such as screws.

Conventional metal shells have a wall thickness of 2-8 mm, in order to be able to counteract warping of the metal shell upon insertion into the bone, which can lead to deformation of the metal shell. This makes it significantly more difficult to introduce an insert or an annular insert. If the preferably annular insert according to the invention is already inserted into the metal shell before implantation, i.e. pre-mounted, the metal shell can be designed so as to be thinner. As a result, metal shells having a reduced thickness of less than 3 mm, preferably less than 2 mm, but at least 1 mm, preferably 1.5 mm, are possible. The mounted (preferably ceramic) annular insert forms a stable bond with the shell which counteracts deformation or warping of the metal shell upon insertion. The annular insert holds the metal shell in shape. The specific arrangement of the bores in the metal shell makes it possible, even in the case of a mounted annular insert, for the implant to be fastened using fastening means, preferably screws.

An annular insert is preferably mounted by an outer surface, in a shell prior to delivery to a user, i.e. connected to the shell in a form-fitting, preferably force-fitting, particularly preferably frictional, manner. The annular insert is therefore held in position in the shell, preferably in the metal shell, by means of a frictional connection or a press fit.

Implants according to the invention are modular and can have different sizes of outside dimensions at the same inside geometry. The inserts of the implants according to the invention can also be produced having different outside dimensions at the same inside geometry. As a result, it is possible for inserts of different sizes to be frictionally connected, preferably by means of a clamping surface, to shells of different sizes. In this case it is important for the clamping surface of the insert to be operatively connected to the clamping surface of the shell, and a force-fitting connection is possible. A modular system consists of shells of different dimensions and inserts of different dimensions. The selection is then made depending on the diameter of the head of the prosthesis to be inserted, and the geometry of the patient's joint which is intended to be replaced by the implant and the head of the prosthesis.

In one embodiment of an implant according to the invention, the insert ends flush with the shell in the direction of the first region. In another embodiment the insert protrudes beyond the shell in the direction of the first region but, in this respect, is frictionally connected to the shell for example by means of a clamping surface, such that a secure fit is ensured.

In a particularly preferred embodiment, an implant according to the invention additionally comprises a second shell, a bipolar shell. Said bipolar shell is arranged between the insert and the first shell. A bipolar system thus results. The sphere of the prosthesis head is arranged in the insert and is in moving connection therewith. The second shell is movably arranged between the insert and the first shell. The insert is connected to the bipolar shell n a form-fitting or force-fitting, preferably frictional, manner. This special arrangement increases the freedom of movement and significantly reduces the risk of dislocation. The sphere of the prosthesis head is movably arranged in the insert, and in addition the second shell is movably arranged in the first shell. As a result of this arrangement, two pivot points result. A first pivot point about which the sphere moves, and a second pivot point about which the second shell moves. This increases the angle of mobility of the joint. An expanded possibility for rotation results.

In this case, in one embodiment the second shell is formed of metal, ceramic or plastics material, preferably plastics material, particularly preferably polyethylene. The wall thickness W_{B} of a bipolar shell consisting of plastics materials is 6-10 mm.

The insert, preferably the annular insert, is form-fittingly and/or force-fittingly connected to the bipolar shell. As a result, quasi an integral part consisting of the shell and insert is formed.

In a further embodiment of an implant according to the invention, the second shell comprises a receiving chamber which can receive the insert, preferably the annular insert. The receiving chamber of the shell, and the outside dimensions of the insert, are matched to one another such that, upon mounting, a form-fitting and/or force-fitting connection can be established between the shell and the insert. The inner surface of the bipolar shell is matched to the outside shape of the insert and can be shaped accordingly. The receiving chamber comprises a surface which limits the introduction of the insert. In the mounted state, the surface of the receiving chamber and the base surface adjoin one another. The second shell can comprise regions of different wall thicknesses. The outside of the second shell is preferably hemispherical, and as a result the movability between the second and first shell is ensured.

In a further embodiment, the insert is introduced into the plastics shell and protrudes therefrom in the direction of the first region.

In a particular embodiment of an implant according to the invention, the two pivot points, first and second pivot point, are arranged at a distance from one another. The two pivot points are preferably located in the lengthening of the axis of rotation, but can also be arranged in a line in parallel with the axis of rotation. The distance a between the two pivot points is between 0.1 mm and 5 mm, preferably between 1.5 mm and 2.5 mm. If the two pivot points are arranged so as to be offset, the radius, measured from the first pivot point to the outside of the second shell, changes continuously. This radius change can result from an increase in the wall thickness, proceeding from the smallest radius. In this case, both the wall thickness of the second shell and/or the wall thickness of the insert can change.

In one embodiment, the inner surface of the bipolar shell is formed in a manner deviating from a hemispherical shape. The sphere slides in the insert and not in the bipolar shell, and touches only the sliding surface of the annular insert. The outside of the bipolar shell is preferably hemispherical, and slides in the first shell. In one embodiment, the geometry of the inside of the shell, in which the bipolar shell slides, is hemispherical, and in another embodiment the inside geometry follows the rules of the insert according to the invention.

In a preferred embodiment, an annular insert is mounted, by means of the outside, in the bipolar shell, prior to implantation, for the purpose of use in a bipolar system. This results in a pre-mounted implant.

In a preferred embodiment, the sphere of the prosthesis head also consists of ceramic. Since, in the system according to the invention, this slides in the preferably ceramic insert, a ceramic-ceramic tribological pairing results on the sphere head side. In the case of conventional bipolar systems in which the sphere of the prosthesis head is received in a bipolar shell consisting of plastics materials, this interface is to be considered critical with respect to the wear, since a ceramic-plastics material tribological pairing results there. In a system according to the invention the friction during use is significantly reduced, in the largely moving articulation surface, and the entire system exhibits significantly lower wear.

Advantages are:
a lower height of the implant in the annular embodiment, and therefore a very small recess is required, e.g. in the bone. This allows for implantation in the case of small or low bone stock.
no punctual loads, but rather linear loads having reduced maximum values, similar to the physiological load bearing capacity.
compared with a hemispheric insert, the geometry according to the invention creates a reduced pressure on the contact point.
the insert having the geometry according to the invention can be combined, without restriction, with conventional shells and spherical sliding partners.
the changed geometry does not have a disadvantageous effect on the production costs, since known production methods can be used.
material is saved and, during production and wear, as well as a reduction in the volume of an annular insert (e.g. useful space in process plants), as a result of which there is a cost advantage.

The invention describes an implant for tribological pairing in endoprosthesis, comprising at least one shell, into which a preferably ceramic insert is introduces. The insert comprises an outside and an inside, and is provided with a sliding region on the inside for receiving a spherical sliding partner, and a surface on the outside for fastening in the shell (4, 14).

In order to minimize the friction between the spherical sliding partner and the insert, an adjusted inside geometry of the implant is proposed.

In summary, the (preferably ceramic) insert (1) for the tribological pairing with a spherical sliding partner (5) is designed as a half shell or in an annular manner, and comprises an inner surface which is formed as the sliding region (2) for receiving a spherical sliding partner (5). The sliding region (2) corresponds to a portion of half a spindle of a spindle torus, in the longitudinal extension. The height H_{G} of the sliding region (2) corresponds to 20-80% of the diameter of the sphere to be inserted, and preferably 50-95% of the height of the implant.

The implant (1) preferably comprises a first region for introducing the sliding partner, and a second region that limits the reception of the sliding partner. Furthermore, the implant comprises an inner surface which is designed as the sliding region (2) for receiving a spherical sliding partner (5), an outside (6), on which a clamping surface (3) is arranged at least in part, by means of which the annular insert (1) can be fastened in a shell (4), an end face (10) which represents the transition from the inside to the outside in the first region, and a base surface (9) which is located opposite the end face (10) in the second region. The sliding region (2) of the implant corresponds to a portion of half a spindle of a spindle torus in the longitudinal extension.

Advantageous embodiments of the annular insert according to the invention are described in the figures, in which:

FIG. 1a: is a side view of an annular insert,

FIG. 1b: is a cross section showing the annular insert according to FIG. 1a,

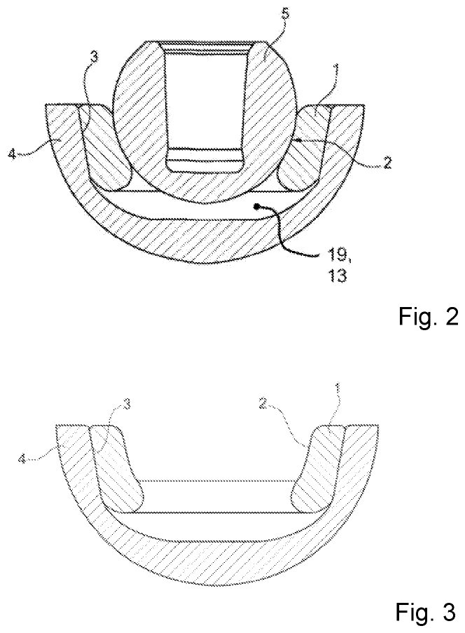

FIG. 2: shows an embodiment of an annular insert according to the invention,

FIG. 3: is a cross-sectional view of an implant according to FIG. 2

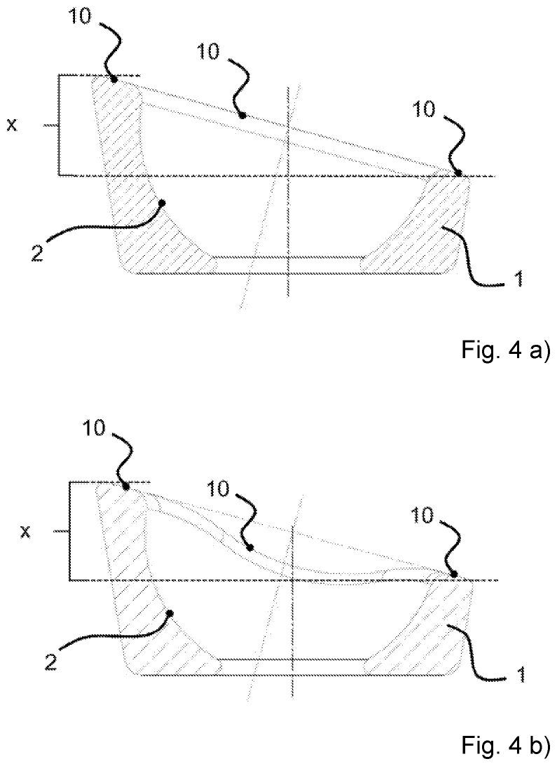

FIG. 4a: shows an embodiment of an annular insert

FIG. 4b: shows a further embodiment of an annular insert

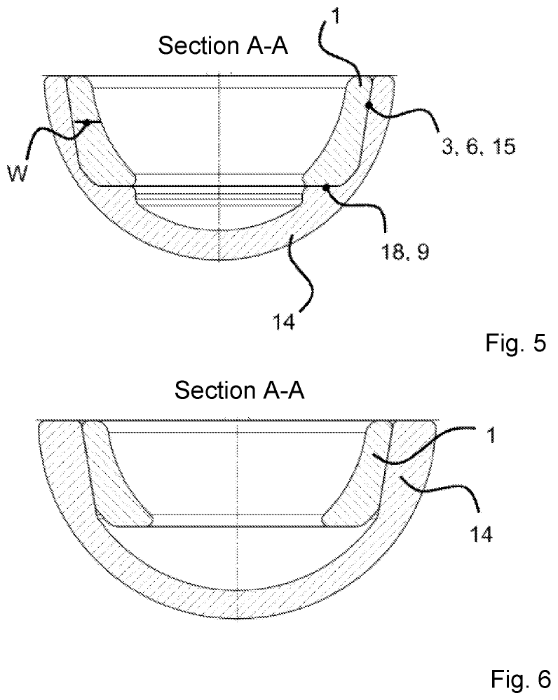

FIG. 5: is a cross-section of an example of an implant according to the invention

FIG. 6: is a cross-section of a further embodiment of an implant

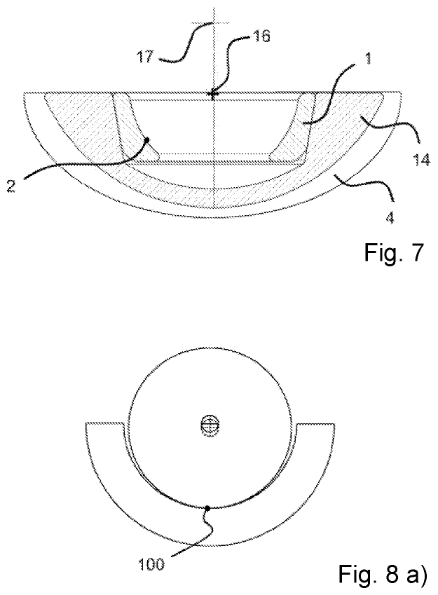

FIG. 7: is a cross-section of a further embodiment of an implant according to the invention

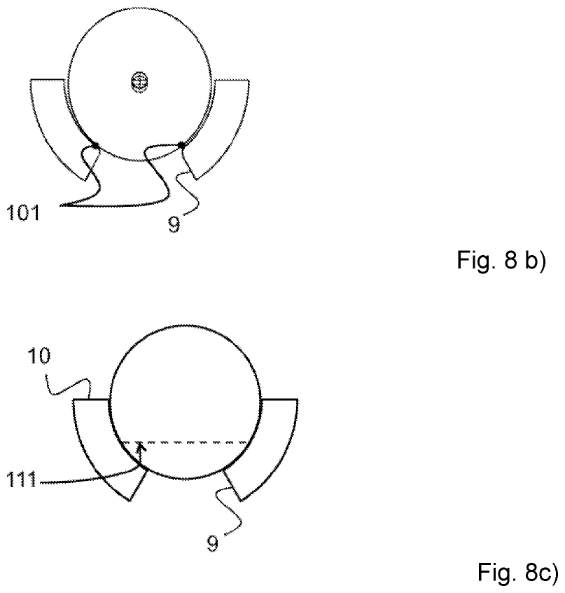

FIG. 8: shows the contact points of the spherical sliding partner in a conventional insert (A), a conventional annular insert (B), and the implant according to the invention (C) in the annular embodiment thereof,

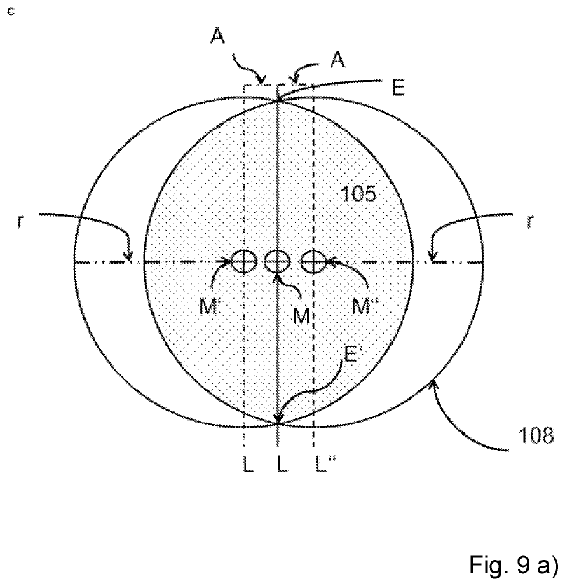

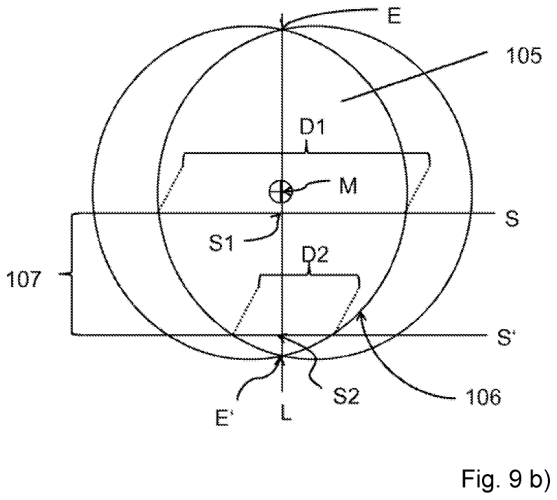

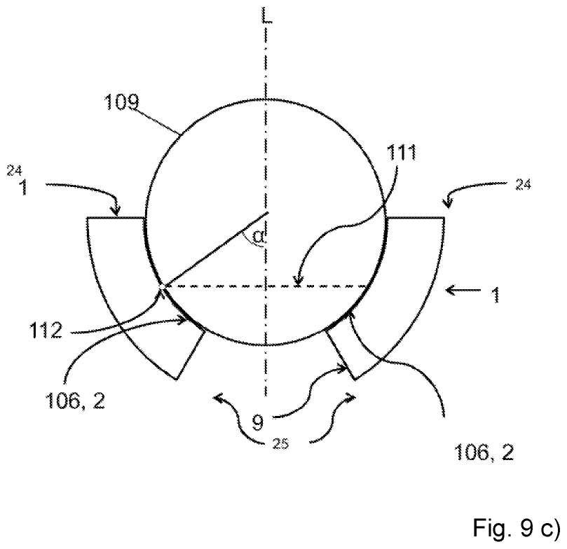

FIG. 9a-c): shows the geometry of the spindle,

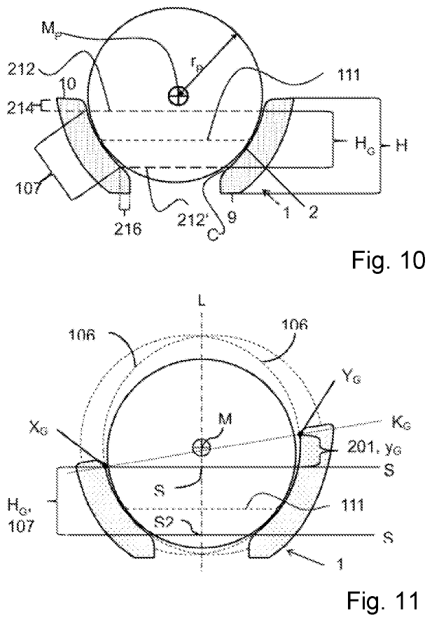

FIG. 10: shows a preferred embodiment of the implant according to the invention,

FIG. 11: shows a preferred embodiment of the geometry of the cranial elevation,

FIG. 12: shows a preferred embodiment of the geometry of the cranial lengthening.

### LIST OF REFERENCE SIGNS AND ABBREVIATIONS

1 insert
2 sliding region
3 outer surface, surface, clamping surface
4 shell
5, 109 head, sphere of the prosthesis
6 outside
7 clamping surface
8 implant
9 base surface
10 end face
13 recess
14 second shell, bipolar shell
15 receiving chamber, pocket
16 first pivot point
17 second pivot point
18 surface
19 free space
20 elevation
100 contact point
101, 112 annular contact, contact line
105 spindle
106 outer surface of the spindle
107 portion of the spindle
108 circle describing the spindle torus
110 tangent point
111 sectional plane of the contact line
112 contact line
201 region of the cranial elevation of the sliding region
202 region of the cranial lengthening of the insert
205 region of the clamping surface (shown in dashed lines)
212, 212′ circle line (orientation aid)
214 infeed zone
216 discharge zone
A distance between the axis of rotation and the center point M of the circle describing the spindle torus
C clearance
D1 maximum diameter of the sliding region, arranged in the first region
D2 minimum diameter of the sliding region, arranged in the second region
E, E′ point of intersection of the spindle outer surface with L
F cross-sectional surface
H height of the insert
H_{G} height of the sliding region
K straight line describing the cranial elevation
K_{G} straight line describing the cranial elevation of the sliding region
K′ straight line describing the cranial lengthening
KG spherical sliding partner
L longitudinal axis of the spindle, axis of rotation
L′, L″ axis, in parallel with L, of the circle, describing the spindle torus, through the center point
M center point of the spindle
M′, M″ center point of the circle describing the spindle torus
M_{P} center point of the spherical sliding partner
r radius of the circle describing the spindle torus
r_{P} radius of the spherical sliding partner (KG, prosthesis head)
R axis of rotation of the clamping surface
S, S′ planes normal to L
S1, S2 points of intersection of the normal planes S, S′ on the longitudinal axis L
X maximum of the insert or cranial elevation in the direction of the end face
X_{G} maximum of the sliding region without a cranial elevation in the direction of the end face
X′ maximum of the insert without a cranial lengthening in the direction of the base surface
x height difference of the cranial elevation
Y maximum of the insert of the cranial elevation
Y_{G} maximum of the sliding region of the cranial elevation
Y′ maximum of the insert of the cranial lengthening
y height difference of the cranial lengthening

An insert 1 according to the invention is shown in FIG. 1a and FIG. 1b, which insert is part of an implant according to the invention. FIG. 1a is a view of said insert 1 and FIG. 1b is a cross-section thereof along an axis of rotation L according to FIG. 1a. The insert 1 comprises an inner portion of a spindle, also referred to as a (non-hemispheric, covalent) sliding region 2 or inner surface. In the case of hip joint prosthesis the prosthesis head 5 is articulated thereon, see FIG. 2. An outer surface, preferably clamping surface, 3 is arranged on the outside 6 of the insert 1, by means of which surface the insert can be anchored in a shell 4, 14. The height H of the insert is shown by dashed lines and extends from the end face 10, the first region, to the base surface 9, the second region. The height is between 5 and 20 mm. The axis of rotation is denoted by L.

FIG. 2 is a cross-sectional view of the insert according to the invention which is formed as an annular insert 1 and is inserted into a shell 4. A prosthesis head 5 is inserted into the annular insert 1. The free space 19, in the form of a recess 13, is visible between the sphere 5, or the sliding partner, and the shell 4.

FIG. 3 is a cross-section of an annular insert 1 according to the invention inserted in a metal shell 4. The annular insert comprises an inner annular portion, a sphere or a hemispherical sliding region 2. A clamping surface is denoted by reference sign 3. The clamping surface 3 can be designed so as to be circumferential, and thus correspond to the size of the outside 6 of the annular insert 1. In a manner deviating therefrom, the clamping surface 3 can include only portions of the outside 6 and be of different shapes. It is also possible for recesses or interruptions (not shown) to be present in the clamping surface 3.

FIG. 4a) shows an annular insert 1 having a cranial elevation which is achieved a continuous incline of the first region, the end face 10 of the annular insert 1, and which is of height x. In this case, the center point of the sliding surface 2 is located on the plane formed by the end face 10.

FIG. 4b) shows an annular insert 1, the cranial elevation of which is achieved by a balcony-like protrusion or a shaped projection of height x, the inside of the cranial elevation being a continuation of the sliding region 2, of the hemispherical receiving chamber or the inside 2 of the annular insert 1.

FIG. 5 shows an annular insert 1 which is introduced into a receiving chamber 15, a pocket of a second shell 14. The inside shape of the receiving chamber 15 corresponds to the outside shape of the annular insert 1. Both shapes are matched to one another such that the annular insert 1 can be received in the receiving chamber 15 of the second shell 14 in a force-fitting and/or anti-turn manner. In this case, the receiving chamber 15 comprises a surface 18 which limits the introduction of the annular insert 1. In the mounted state, the surface 18 of the receiving chamber 15 and the base surface 12 adjoin one another.

FIG. 6 shows an annular insert 1 which is introduced into a second shell 14 in a force-fitting manner, said second shell 14 not comprising any means for limiting the insertion depth of the annular insert 1.

FIG. 7 shows an implant according to the invention which comprises an annular insert 1, a second shell 14, and a shell 4. The center point 16 of the inside 2 of the sliding region of the annular insert 1, the first pivot point 16, is arranged so as to be at a distance from the second pivot point 17 of the shell 14.

FIG. 8a) schematically shows the most likely locations of friction of a conventional insert, FIG. 8b) shows this for a conventional annular insert, and FIG. 8c) shows this for an implant according to the invention comprising an annular insert. In the case of a known semicircular insert, the contact point 100 is positioned between the insert and KG, on the base of the insert. In the case of a known annular insert, the contact 101 (FIG. 8b) is located between the insert and the KG, on a line 101. This is preferably a linear contact, and linear friction. This line 101 is arranged in the region close to the base surface 9. In the insert according to the invention, the correspondingly designed geometry means that the contact line is arranged on the plane 111, at a distance from the base surface 9 in the direction of the end face 10 (FIG. 8c).

FIG. 9 shows the determination of the inside geometry of the insert according to the invention. The spindle torus 105 in FIG. 9a) is described by a circle 108 having a radius r that has a center point M′/M″ and rotates about the axis of rotation that corresponds to the longitudinal axis L of the spindle. The axes L′ and L″ are in parallel with L and extend through M′, M″. The distance between L′/L″ and L is smaller than the radius r. The spindle intersects the longitudinal axis in the points E and E′.

In FIG. 9b) the determination of the portion 107 is made clear. The portion 107 of the spindle is located in a half 105 and is formed by the planes normal to L (S and S′). Said planes intersect L in points S1 and S2 and in this case the following applies: S1=M or S1 is located between M and E′, S2=E′ or S2 is located between S1 and E′. Both the points of intersection S1, S2 are therefore located in half of the spindle and do not exceed the center thereof. The diameter D1 in the first region is larger than the diameter D2 in the second region, D1 being larger than the diameter of the KG to be inserted. D2 is smaller than the diameter of the KG to be inserted, as a result of which (in the case of an annular insert) the KG is prevented from falling out.

FIG. 9c) shows the sectional plane 111 of the contact line 112 between the KG 109 and the implant 1, on the sliding surface 2 thereof, according to the outer surface of the spindle 106.

The contact line 112 corresponds to a sectional plane 111 on the spherical sliding partner 109. Owing to the spindle shape, the region of the end face 10 is inclined towards the spherical sliding partner or towards the longitudinal axis L. As a result, the diameter D1 has a smaller value compared with a diameter of a comparable hemispheric sliding region measured at the same point. As a result, the contact line 112, on which the spherical sliding partner 109 moves, is displaced towards the end face 10 of the insert and away from the base surface 9.

FIG. 10 shows the height H_{G} of the non-hemispherical sliding region 2, shown on an annular insert 1 having KG inserted having a center point M_{P} and a radius r_{P}. The sliding region 2 corresponds to a portion 107 of half a spindle of a spindle torus, in the longitudinal extension. The circle lines 212, 212′ are used merely for orientation. The portion 107 is limited by the infeed zone 214 in the region of the end face 10, and by the discharge zone 216 in the region of the base surface 9. The infeed zone 214 and the discharge zone 216 are not part of the sliding region 2 and therefore do not necessarily follow the spindle geometry. The clearance C corresponds to the formula C=(r−r_{P})*2. The KG slides on the sliding surface 2 on the circle line described by the plane 111.

FIG. 11 shows the region of the cranial enlargement of the sliding region 201. The height y_{G} of the cranial enlargement extends between a point of intersection of the normal plane S with the end of the sliding region 2 in the direction of the infeed zone 214 and the point Y_{G}. In this case, the point Y_{G} lies on a straight line K_{G} that intersects L. The straight line K_{G} extends between the point of intersection X_{G} of the normal plane S with the end of the sliding region 2 on the outer surface of the spindle 106 and the point Y_{G}. In this case, the points X_{G} and Y_{G} are arranged on a plane which extends through the end points of the sliding region 2. The two points X_{G} and Y_{G} are mutually spaced. If the cranial elevation is symmetrical, i.e. the ascent and fall are of the same length and each extend over 180°, then the point X_{G} is arranged opposite the point Y_{G}. It is then 180° away from the point Y_{G}. In the case of an embodiment of this kind, a gentle ascent of the cranial elevation can be achieved. If the ascent or the fall of the cranial elevation are steeper, two points X_{G} may be provided. The slope of the cranial elevation begins and ends at these points. Between these two points X_{G}, where no cranial elevation is formed, the insert can be formed so as to be planar and flat, without any elevation or depression. In the preferred embodiment shown, the straight line K_{G} also intersects the center point of the spindle, and Y_{G} lies on the outer surface of the spindle. For the height H_{G} of the sliding region of an insert having a cranial elevation, the following applies: H_{G}′=H_{G}+y. The same relations can be created for the cranial enlargement of the insert, proceeding from the height of the insert.

FIG. 7 shows the region of the cranial lengthening 202 of the insert. The region results between the point Y′ on a straight line K′ and the sectional plane S′. The straight line K′ extends from point X′, which is located on the plane S′ and the outer surface of the spindle 106, to a further point Y′ which is located opposite X′ and represents the maximum of the cranial elevation. In this case, X is located on the opposite side from Y′, i.e. a straight line from X′ to Y′ intersects L. For the height of the implant the following applies: H′=H+x. The region 205 corresponds to the clamping surface of the insert. As shown, this region is preferably in parallel with the straight line K′ which shows the maximum dimension of the insert in the region of the base surface. The axis of rotation R of this clamping surface is therefore perpendicular to the straight line K′. An insert of this kind then appears as an insert having a cranial elevation, the inside geometry of which is tilted away from the cranial elevation in the form of a portion of a spindle.

1. Insert for the tribological pairing comprising a spherical sliding partner, the insert being formed in a half shell or annular manner and comprising an inner surface which is formed as a sliding region for receiving a spherical sliding partner, wherein the sliding region corresponds to a portion of half a spindle of a spindle torus in the longitudinal extension, the height H_{G} of the sliding region corresponding to 20-80% of the diameter of the sphere to be inserted and/or 50-95% of the height H of the implant, and the maximum diameter D1 of the sliding region being larger than the diameter of the spherical sliding partner to be inserted.

2. Insert according to claim 1 for the tribological pairing, comprising
an outside (6), wherein a clamping surface is arranged on the outside, at least in part, by means of which the annular insert can be fastened in a shell, and
a first region for introducing the sliding partner, and
a second region which limits the reception of the sliding partner.

3. Insert according to either claim 1, wherein the insert is annular and the minimum diameter D2 of the sliding region is smaller than the diameter of the spherical sliding partner to be inserted.

4. Insert according to claim 1, wherein the insert has an axis of rotation R, and wherein the clamping surface of the annular insert is arranged at an acute angle of 10°-20° relative to the axis of rotation R, such that the outside dimensions of the insert in the second region are smaller than in the first region.

5. Implant according to claim 4, wherein the angle of the clamping surface is 18°-18.5°.

6. Insert according to either claim 4, wherein the axis of rotation R is arranged so as to be in parallel with the axis of rotation L, preferably corresponds to the axis of rotation L.

7. Insert according to claim 2, wherein the clamping surface (3) comprises recesses in the form of notches or tangential cuts.

8. Insert according to claim 1, wherein the radius of the circle r describing the spindle torus, the clearance C, and the radius of the sphere of the prosthesis r_{P} are in the relationship according to Formula I.
C=(r−rₚ)*2 (Formula I)

9. Insert according to claim 1, wherein the following applies: 10 μm<C<500 μm.

10. Insert according to claim 1, wherein the insert is ceramic.

11. Insert according to claim 1, wherein the annular insert, preferably the sliding region, is enlarged cranially.

12. Implant comprising at least one shell and an insert according to claim 1.

13. Implant according to claim 12, wherein the shell is made of metal and has a wall thickness of from at least 1 mm to less than 3 mm, preferably less than 2 mm.

14. Implant according to either claim 12, comprising at least two shells, wherein the insert is inserted into a second shell and the second shell is inserted into the first shell.

15. Implant according to claim 13, wherein the second shell is made of plastics materials, preferably polyethylene.
